(19) United States
(12) Patent Application Publication
(10) Pub. No.: US 2020/0278300 A1
(43) Pub. Date: Sep. 3, 2020

(54) DEVICE FOR AUTOMATIC SPECIES ANALYSIS AND METHOD FOR CARRYING OUT THE SAME

(71) Applicant: HELMHOLTZ-ZENTRUM FÜR UMWELTFORSCHUNG GMBH - UFZ, Leipzig (DE)

(72) Inventor: Susanne DUNKER, Leipzig (DE)

(21) Appl. No.: 16/648,650

(22) PCT Filed: Oct. 6, 2017
(86) PCT No.: PCT/EP2017/075553

§ 371 Date: Mar. 18, 2020

(51) Int. Cl. G01N 21/64; G01N 15/02; G01N 33/00; G06K 9/00

(52) U.S. Cl. CPC G01N 21/6486; G01N 21/6456; G01N 15/0205; G01N 2021/635; G06K 9/00134; G06K 9/00147; G01N 2021/6419; G01N 33/0098

(57) ABSTRACT

The invention relates to a device for automatic species analysis and to a method for carrying out the same. In particular, the invention relates to a device for automatic species analysis within a large biological group and to a method for automatically carrying out such analyses. A device according to the invention for automatic species analysis within a large biological group comprises a first beam path (10), directed from a first laser radiation source (12) to a sample region (40), the fluorescence radiation emitted by a sample (42) within the sample region (40) as a result of excitation by the first laser radiation source (12) being collected and fed to a first spectrometer (14) for spectral evaluation; a second beam path (20), directed from a second laser radiation source (22) to the sample region (40), the fluorescence radiation emitted by the sample (42) within the sample region (40) as a result of excitation by the second laser radiation source (22) being collected and fed to a second spectrometer (24) for spectral evaluation; and a camera (30), which is designed to capture a photograph of the sample (42) within the sample region (40); the device also comprising a means for automatic species analysis (50), which is designed to carry out an automatic species analysis from the sample-related results of the spectroscopic evaluation of the first spectrometer (14) and of the second spectrometer (24) and the photograph of the camera (30) by means of a multi-step threshold value comparison for certain determination parameters stored in an associated database. A method according to the invention serves to carry out an automatic species analysis by means of a device according to the invention.

The present invention relates to a device for automatic species analysis and a method for carrying out the same. In particular, the present invention relates to a device for automatic species analysis within a biological greater group and a method for automatically carrying out such analyses.

### PRIOR ART

For the examination of environmental samples, in particular those taken from water or air, the occurrence and distribution of certain biological species, in addition to an analysis of their chemical composition, is a characteristic feature for the assessment of the quality and purity of the samples as well as the current state of the biological system sampled.

If, for example, the ecological status of European waters is considered, it becomes clear that in many parts of the EU and also in Germany a large proportion of water bodies with less than good ecological potential are assessed. Phytoplankton, among others, is assessed as a biological quality component. Phytoplankton are microscopically small plant organisms, which as primary producers are at the bottom of the food chain. A disturbing change within the phytoplankton community has far-reaching consequences on the rest of the food chain and thus on the entire ecosystem. Due to the rapid growth of phytoplankton organisms compared to higher aquatic plants, phytoplankton is a particularly good indicator of short-term changes, such as nutrient inputs. It is assumed that the greatest burden for lakes is caused by excessive nutrient inputs from the respective drainage areas, for example in agricultural areas. Especially nitrogen and phosphorus are crucial for phytoplankton-growth, as they are needed in large quantities for growth. A high external nutrient input leads to massive growth of phytoplankton organisms, changes in the composition of the phytoplankton and, in the worst case, to an impoverishment of biodiversity and mass development of some species, which is closely related to health hazards and the “collapse” of waters. Here, a group of phytoplankton organisms is in particular focus: cyanobacteria. These organisms are enormously adaptable and can occur even under the most difficult conditions, such as lack of light and nitrogen limitations. It is known that many cyanobacteria species produce toxins that have neuro-, cyto-, dermato- and hepatotoxic effects and therefore represent a major health hazard for drinking water or leisure use.

In recent years, a number of efforts have been made to counteract the formation of cyanobacterial masses. In particular, efforts have been made to reduce the nutrients required for phytoplankton growth. Several examples show that a reduction of nutrients alone is not sufficient to limit the massive growth of this phytoplankton group, but that other effects have a promoting effect. This shows that our understanding of the controllability of biological components is not yet sufficient to be able to have a controlling effect. In the lake ecosystem there is a highly complex interaction of different influencing factors which are in close interaction with each other.

Therefore, a continuous recording of the phytoplankton composition is necessary for a reliable assessment of the water quality. Up to now, this has generally involved sampling followed by microscopic assessment. However, it has been shown that this sampling only covers a tiny part of the development of the phytoplankton during the course of the year and that there is a very high variability of the composition only in the course of a day or a few days. Sampling once a month reflects a random condition that can vary extremely within a few days. For example, a massive occurrence of cyanobacteria may occur in the interim period of the sampling interval and then result in a different assessment.

For an actual understanding of phytoplankton dynamics, a much shorter sampling interval is necessary, but there are massive limitations due to the immense time required for microscopic analysis. According to the service specifications for limnology of the German Society for Limnology, the evaluation of a single sample alone takes 60-180 minutes, depending on the effort and depth of determination of the organisms (Service specifications for limnology, German Society for Limnology e.V., 2012). This analysis must be carried out by a trained taxonomist. Furthermore, this time frame only records how many organisms of a species or genus are present, but not, for example, what the cell size distribution looks like or how many reserve substances are present per cell. However, these statements have great relevance against the background of a scientific consideration. Since cell size provides information on the physiological activity of the species, cell size distribution is particularly important for estimating the growth potential of phytoplankton species in the water or, for example, for process control of algae plants.

Another example where biological markers can be used to assess the quality of the environment are studies on air purity. For allergy sufferers, for example, a reliable assessment of the current pollen count in the air is particularly important. However, since the count can vary greatly depending on the region and distribution vector, these studies also require spatially and temporally close sampling. In particular, however, it is important to evaluate the environmental samples taken as soon as possible. Up to now, these evaluations have also been carried out mainly by taxonomists who have been trained accordingly, which also makes these investigations very expensive and time-consuming.

### DISCLOSURE OF THE INVENTION

It is therefore one of the purposes of the present invention to specify a device for automatic species analysis and a method for carrying out the same which overcomes the disadvantages of the prior art described. In particular, a device for automatic species analysis according to the invention should be capable of obtaining information about the presence, distribution and specific properties of biological species in a sample by means of a meteorological evaluation without the participation of a taxonomist. The use of a completely automated method for species analysis is intended to reduce the examination costs and the time required for the examinations.

According to the invention, these tasks are solved by the features of the independent claims 1 and 7. Suitable features of the invention are contained in the respective sub-claims.

An device according to the invention for automatic species analysis within a biological greater group comprises a first beam path directed from a first laser radiation source onto a sample area, whereby the fluorescent radiation generated by a sample (biological sample material, e.g. phytoplankton or a pollen) within the sample region by excitation by the first laser radiation source is collected and fed to a first spectrometer for spectral evaluation; a second beam path from a second laser radiation source directed to the sample region, wherein the fluorescent radiation emitted by the sample within the sample region by excitation by the second laser radiation source is collected and fed to a second spectrometer for spectral evaluation; and a camera adapted to take a photographic image of the sample within the sample area; the apparatus further comprising means for automatic species analysis adapted to select from the sample-related results of the spectroscopic evaluation of the first spectrometer and the second spectrometer (d. (i.e. first and second fluorescence spectrum of the sample) and the photographic image taken by the camera by means of a multi-stage threshold value comparison for specific determination parameters stored in an associated database, to perform an automatic species analysis.

Species analysis within a biological greater group means that the species to be automatically identified and examined with regard to their specific properties (i.e. the biological sample material contained in an environmental sample) belong to a certain biological greater group. A greater group may be, for example, certain species of phytoplankton or a pollen group. However, a device according to the invention can generally be designed for automatic species analysis of a large number of biological greater groups.

In the first beam path, the radiation of a first laser radiation source is directed as excitation radiation onto a sample region, the fluorescence radiation emitted by a sample within the sample region by excitation by the first laser radiation source being collected, e.g. by means of appropriate collecting optics, and fed to a first spectrometer for spectral evaluation, i.e. for producing a spectrum of the first fluorescence radiation. This spectrometer can be a grating spectrometer, a prism spectrometer or a filter spectrometer, for example. In particular, the spectrometer can be designed as a filter stack with a variety of dichroic filters. The spectral measurement is preferably carried out by a corresponding camera (e.g. line camera or matrix camera), whereby a spectral assignment to individual spectral ranges is made via the pixel positions. Preferably, spectral ranges are combined to individual channels (e.g. by numbered channels with a spectral width of 50 nm).

In the second beam path, the radiation of a second laser radiation source is directed onto the sample area as excitation radiation, the fluorescence radiation emitted by the sample within the sample area by excitation by the second laser radiation source being collected, e.g. by means of appropriate collecting optics, and fed to a second spectrometer for spectral evaluation, i.e. for producing a spectrum of the second fluorescence radiation. This spectrometer can be a grating spectrometer, a prism spectrometer or a filter spectrometer, for example. In particular, the spectrometer can be designed as a filter stack with a variety of dichroic filters. The spectral measurement is preferably carried out by a corresponding camera (e.g. line camera or matrix camera), whereby a spectral assignment to individual spectral ranges is made via the pixel positions. Preferably, spectral ranges are combined to individual channels (e.g. by numbered channels with a spectral width of 50 nm).

The guidance of the first and second beam paths onto the sample can be collinear or independent (non-collinear). Preferably, both beam paths are directed to a common position or two closely spaced positions, preferably less than 10 μm, more preferably less than 5 μm, more preferably less than 2 μm, more preferably less than 1 μm and even more preferably less than 0.5 μm apart, in the sample area. Preferably, the fluorescence radiation from the excitation of a sample via the first beam path is fed exclusively to the first spectrometer for spectral evaluation. Preferably, the fluorescence radiation from the excitation of a sample via the second beam path is fed exclusively to the second spectrometer for spectral evaluation.

Preferably, the emission wavelength of the first laser radiation source and the emission wavelength of the second laser radiation source differ from each other. In particular, a wavelength difference between the wavelengths of the laser radiation sources may be at least 10 nm, more preferably at least 25 nm, more preferably at least 50 nm, more preferably at least 100 nm, and even more preferably at least 250 nm. Preferably, the emission wavelength of the first laser radiation source is between 480 nm and 500 nm. Also preferred is that the emission wavelength of the second laser radiation source is between 550 nm and 570 nm. In particular, the emission wavelength of the first laser radiation source is preferably 488 nm. Especially preferred is the emission wavelength of the second laser radiation source at 561 nm.

The camera used to photograph the specimen within the specimen area may preferably be a still or video camera to visually capture the specimen within the specimen area. The camera may also include an optical imaging system to magnify the image (a so-called microscopy assembly). Alternatively, the camera can also be designed as an element of the spectroscopy system. In particular, a spectrometer camera can also be used to take photographs of the sample within the sample area. Photographic images of the sample can also be taken simultaneously in several spectral ranges, e.g. by means of a filter spectrometer, in which the spectrometer camera is illuminated simultaneously via different color filters aligned side by side. Preferably, a spectrometer camera is configured to simultaneously take a large number of fluorescence microscopic images, each covering different wavelength ranges. In particular, a spectrum can consist of a spectrally resolved series of individual fluorescence microscopic images of a sample (i.e. simultaneous generation of spectrally and locally resolved fluorescence microscopic images by a spectrometer).

A device according to the invention further comprises a means for automatic species analysis, whereby an automatic species analysis for a sample is carried out from the sample-related results of the spectroscopic evaluation of the first spectrometer and the second spectrometer (e.g. the fluorescence spectra as measured spectral intensity over wavelength) and the photographic image of the camera. The spectroscopic data of both beam paths are therefore evaluated in combination with at least one photographic image for species determination within a biological greater group.

The means for automatic species analysis accesses an associated database, which contains certain determination parameters for an automatic species analysis. The individual determination parameters are marked by threshold values and show a multilevel order towards an increasingly fine taxonomic differentiation (“decision tree”). Multi-level means that the determination parameters in the database preferably have an order from general group membership, family level to species determination. Preferably, the database of the means for automatic species analysis contains additional determination keys for species analysis, e.g. from metadata on the origin of the environmental sample, probability assumptions on the presence of certain species or other limiting and distinguishing features.

The means for automatic species analysis can then perform an automatic species analysis by means of a multilevel threshold comparison corresponding to the multi-level order of the database. In particular, a taxonomic differentiation of spectral groups (evaluation of the sample-related spectroscopy data), a biological family level (evaluation of the photographic image of the sample) up to the species level (further evaluation of the photographic image of the sample) can be performed. Threshold value comparison here means that for a certain determination parameter a threshold value can be stored in the database, which is compared with a corresponding value from the sample-related results of the spectroscopic evaluation of the first spectrometer and the second spectrometer as well as the photographic image of the camera. A differentiation is then made based on whether the value from the sample-related results is above, below or on the threshold value.

The invention is based on the knowledge that a combination of spectroscopic methods with means of automatic image recognition (e.g. by means of deep learning) is particularly suitable for an automatic species analysis. By exciting the fluorescence at two different wavelengths, different excitation paths in the fluorescence of a sample can be evaluated. This allows, in particular, the specific orientation of an arrangement according to the invention to a certain biological greater group by selecting suitable excitation wavelengths. The excitation wavelengths can be selected so that very specific molecules or sample components can be excited to fluoresce. The spectroscopic data obtained can then already be used for an initial classification into spectral classes. By evaluating the photographic image, a further classification at family level can then be made, e.g. based on the geometric dimensions of a sample. A further differentiation at species level can also be made by evaluating the photographic image and in particular by considering additional metadata as a further determination key, especially regarding the expected species distribution based on the origin of the sample.

Preferably, a device according to the invention also has a means for archiving, configured to archive the results of the spectroscopic evaluation of the first spectrometer and the second spectrometer as well as the photographic image of the camera. In this context, archiving means that for a large number of examined samples, the sample-related results of the spectroscopic evaluation of the first spectrometer and the second spectrometer and of the photographic image of the camera can preferably be provided with a time stamp and stored as a complete or reduced data record. In particular, this makes the results of an automatic species analysis available for review at a later time. Such archiving can be used in particular to optimize the threshold values stored in the database for the determination parameters or can be used for additional random checks by a taxonomist.

Preferably, a device according to the invention also has a means for statistical evaluation, configured to carry out a statistical evaluation of a large number of automatic species analyses on different samples. The means for statistical evaluation can be a computer-based statistical evaluation program.

Another aspect of the present invention concerns a method for automatic species analysis comprising the following steps: providing a device for automatic species analysis in accordance with the invention; introducing a sample into the sample region; producing a sample-related spectroscopic evaluation of the fluorescence radiation generated with the first spectrometer and the second spectrometer and a photographic image of the sample with the camera; evaluating the sample-related results of the spectroscopic evaluation of the first spectrometer and the second spectrometer and of the photographic image of the camera by the means for automatic species analysis, wherein an automatic species analysis is carried out by a multi-stage threshold value comparison for specific determination parameters stored in an associated database. For the threshold value comparison, a value for a determination parameter is first determined from the sample-related results of the spectroscopic evaluation of the first spectrometer and the second spectrometer as well as the photographic image of the camera, and this value is compared with the threshold value stored in the database for this determination parameter for the purpose of differentiation decision.

A method according to the invention is particularly suitable for performing an automatic species analysis by means of a device according to the invention described in this document. The individual steps of the method are based on a procedural application of the features mentioned in the section on the corresponding design of the device. The explanations given for the individual features or their preferred or optional designs therefore also apply analogously to a corresponding method step.

Preferably, for the evaluation of the sample-related results of the spectroscopic evaluation of the first spectrometer and the second spectrometer as well as the photographic image of the camera by the means for automatic species analysis via the threshold value comparison, a classification according to spectral groups based on the spectral intensity ratios for individual wavelength ranges in the evaluation of the first spectrometer and the second spectrometer is first made, either alone or in combination. Subsequently, a differentiation on family level can be performed based on an automatic image analysis of the photographic image taken by the camera.

Preferably, further differentiation at the species level is then also carried out on the basis of an automatic image analysis of the photographic image of the camera and/or by taking into account additional determination keys for species identification, e.g. on the basis of metadata on sample origin and/or distribution, probability assumptions on occurrence or other limiting and distinguishing features.

Preferably, the means for automatic species analysis additionally performs a biovolume determination based on the sample-related results of the spectroscopic analysis of the first spectrometer and/or the second spectrometer and/or the photographic image of the camera. The biovolume of phytoplankton cells per measured water volume used as a common indication can be determined in particular from the number of species per volume and the cell area of the sample preferably determined from a photographic image of the sample by the means for automatic species analysis.

A biovolume determination can be based, among other things, on the parameters length, width, circularity and aspect ratio, as well as cell or object diameter, and can be calculated using a specific (e.g. a phytoplankton-specific) formula collection (Hillebrand et al. 1999). If the object is approximately circular and has a width to length ratio (aspect ratio) of 0.6-1, for example, the volume of the object can be calculated using the formula for calculating the volume of a sphere: (π/6)*d³, where d corresponds to the diameter of the cell (or other spherical object under investigation). A more ellipsoidal cell can be additionally identified by a value for elongatedness (aspect ratio >1.5) and its volume can be calculated with the formula (π/6)*d²*h, where d is the diameter and h is the length of the cell. With a width to length ratio (aspect ratio) of 0-0.6, a more precise identification of the particle shape must first be made. If the aspect ratio is more than 4 times the length, a threadlike structure can be assumed, which can be calculated using a cylinder ((π/4)*d²*h). Using the calculated volumes, a biovolume determination can be made using the number of objects per volume.

Preferably, the means for archiving is used to archive the sample-related results of the spectroscopic evaluation of the first spectrometer and the second spectrometer as well as the photographic image of the camera. This step serves in particular for a possible documentation of the examination on individual samples. Further advantages of archiving can be found in the corresponding section of the device description.

Preferably, the means for statistical evaluation is used to perform a statistical evaluation of a large number of automatic species analyses on different samples. In particular, a statistical evaluation can be carried out above all with regard to the distribution frequency of certain species in a particular sampling, e.g. in a water body sample.

Another aspect of the present invention concerns a method for automatic pollen determination based on a method for automatic species analysis according to the invention. The method for automatic pollen determination comprises an evaluation of the pollen size, the maximum axial extension and/or the geometric asymmetry ratio (of the pollen) as determination parameters.

A further aspect of the present invention concerns a method for automatic phytoplankton determination which is based on a method for automatic species analysis according to the invention, wherein excitation of the sample takes place at an emission wavelength of the first laser radiation source between 480 nm and 500 nm, wherein excitation of the sample takes place at an emission wavelength of the second laser radiation source between 550 nm and 570 nm for the excitation of phycoerythrin. The emission wavelength of the first laser radiation source is particularly preferred at 488 nm. The emission wavelength of the second laser radiation source is particularly preferred at 561 nm.

Preferably, the method for automatic phytoplankton determination comprises an evaluation of the cell length, the cell width, the maximum axial extent, the geometric asymmetry ratio (of the cell), the cell size, the colony size, and/or the size of refractive intracellular structures of the cell (i.e. the sample) as determination parameters.

The method for automated phytoplankton determination allows the phytoplankton composition to be determined in natural water samples, for example. The special laser configuration with excitation at 488 nm and 561 nm is particularly advantageous for phytoplankton identification. At an excitation wavelength of 488 nm, ubiqitary carotenoids present in all phytoplankton species are excited, whereas at an excitation wavelength of 561 nm, individual phytoplankton groups can be specifically excited (mainly Bacillariophyta, Cyanobacteria and Cryptophyta).

An evaluation of the sample-related results of the spectroscopic evaluation of the first spectrometer and the second spectrometer as well as the photographic image of the camera by the means for automatic species analysis for automatic species analysis is carried out by a multi-stage threshold value comparison with determination parameters, which are stored in an associated database and can be read out there. A taxonomic differentiation of the phytoplankton species is therefore performed in a multi-stage method in several steps.

In a first step, preferably a division into spectral groups is made based on the sample-related results of the spectroscopic evaluation of the first spectrometer and the second spectrometer. Initially, based on the autofluorescence emission pattern, which results in particular from excitation at 488 nm and 561 nm, a classification into greater phytoplankton groups can be made (e.g. cyanobacteria, green spectral group (Chlorophyta, Euglenophyta), Bacillariophyta, Dinophyta, Cryptophyta, Heterokontophyta).

Subsequently, preferably in a second step, a classification at family level is carried out on the basis of the sample-related photographic image of the camera. A more detailed determination at family level may, in particular, be based on certain geometric or other determination parameters (e.g. cell size, cell shape, structural arrangements within the cells, etc.) that can be extracted from the photographic images by automatic image recognition methods (see Lauterbornia 1997; John; D. M. 2003).

Finally, in a third step, preferably a classification at species level is carried out on the basis of a further evaluation of the sample-related photographic image of the camera with regard to further determination parameters and/or under consideration of additional determination keys (e.g. corresponding probability statements on the species probability based on the sample origin). In particular, information from external sources and databases (e.g. AlgaTerra, GBIF, AlgaeBase, planktonforum.eu) can be used for the determination at species level.

In phytoplankton samples spectroscopically examined according to the invention, the different excitation results in, for example, a characteristic spectral intensity ratio between individual spectral ranges in fluorescent light, by which greater groups of phytoplankton can be distinguished from each other. Each individual measured cell (i.e. a sample) can be assigned to a specific phytoplankton group (cyanobacteria, green spectral group (chlorophyta, euglenophyta), bacillariophyta, dinophyta, cryptophyta, heterocontophyta) by using a coarsely scanned emission spectrum. For excitation at a wavelength of 488 nm, an averaged intensity value can preferably be determined in the spectral range 560-595 nm (referred to as channel 03 without restriction of the generality), in the spectral range 595-642 nm (referred to as channel 04), and in the spectral range 642-745 nm (referred to as channel 05). For excitation at a wavelength of 561 nm, an averaged intensity value can preferably be determined in the spectral range 570-595 nm (referred to as channel 09), in the spectral range 595-642 nm (referred to as channel 10), and in the spectral range 642-745 nm (referred to as channel 11). An excitation at a wavelength of 561 nm allows a specific excitation of phycoerythrin to distinguish particularly relevant cyanobacteria, as well as cryptophyta from the other phytoplankton groups.

Table 1 shows an example of determination parameters as threshold values for a division into spectral phytoplankton groups for a fluorescence excitation at wavelengths of 488 nm and 561 nm for the spectral channel division given in the above example. For this purpose, the spectroscopic intensity values of channel 03 and channel 04 must first be normalized to channel 05 (maximum intensity on channel 05=1 a.u.). Furthermore, channel 09 and channel 10 must be normalized to channel 11 (maximum intensity on channel 11=1 a.u.). The determination parameters can be improved and optimized with stored measurements of different species. If certain thresholds are reached or undercut for certain channels, they can be classified into a spectral group using the information in Table 1. Depending on the necessary requirements for the depth of determination, a phytoplankton composition determined in this way may be sufficient for a determination.

TABLE 1

| Threshold | Channel | Threshold | |
|---|---|---|---|
| comparison | no. | intensity | Result |
| #1 | Channel 04 | >0.6 | Cyanobacteria |
| | Channel 04 | < or =0.6 | further testing |
| | | | on channel 09 |
| #2 | Channel 09 | >0.1 | Cryptophytes |
| | Channel 09 | < or =0.1 | further test on |
| | | | channel 10 |
| #3 | Channel 10 | < or =0.020 | Chlorophytes |
| | Channel 10 | >0.020 and <0.025 | Euglenophytes |
| | Channel 10 | < or =0.025 | further testing |
| | | | on channel 11 |
| #4 | Channel 11 | < or =0.023 | Bacillariophytes |
| | Channel 11 | >0.023 | Dinophytes |

According to the invention, differentiation at family level can be achieved by using measured image information from a photographic image of the sample in addition to the fluorescence emission. With the aid of the image information (e.g. from a brightfield image), starting from an already made spectral assignment to a greater group of phytoplankton (e.g. corresponding to the assignment according to Table 1), a further identification based on various determination parameters (e.g. according to Lauterbornia 1997, John, D. M. 2003) can be made at family level. Important determination criteria, such as the formation of filaments, the number, shape and distribution of chloroplasts, as well as flagellation can be taken from the image information via morphological parameters and evaluated according to a corresponding determination parameter. Here, too, differentiation is preferably carried out by means of a threshold value comparison between threshold values stored in a database and the corresponding values obtained from the image information.

Examples of certain determination parameters that can be evaluated for a differentiation of phytoplankton at family level are filaments (the corresponding threshold value can be determined by the aspect ratio of the cell, filaments are present at aspect ratios <0.3), coccal cells (at aspect ratios >0.3), chloroplast number, chloroplast morphology and the symmetry of the cells.

For phytoplankton families with few families (e.g. Cryptophyta) it is possible to determine individual species by differentiation at family level. For very diverse families further differentiation on species level is necessary. In principle, the taxonomic parameters already mentioned can be used here as well. In addition, further information on morphological characteristics can be used as further determination keys.

As determination parameters at species level, in particular the cell length, the cell width, the maximum axial extension of the cell, its geometric asymmetry ratio, the cell size, the colony size, and/or the size of refractive intracellular structures can be evaluated.

A method for automated phytoplankton determination according to the invention has the advantage over conventional microscopic evaluation:
an enormous reduction of the time required for sample analysis (reduction of the analysis time from approx. 60-180 min (according to the service specifications for limnology of the DGL) to 5-30 min automated recording, therefore the presence of a trained taxonomist is not absolutely necessary) with a statistically better data basis due to a multiple of recorded samples than with microscopic analysis (minimum requirement microscope 400 cells, flow cytometer up to 5000 cells/second), thus even rare species can be recorded;
a much higher objectivity of the evaluation by the automated determination method compared to the subjective assessment by a taxonomist;
greater accuracy of the measurements, since the analyses can be performed at such a high throughput that fixation with a fixation reagent is not necessary; the archivability of the created photographic images for each individual organism and thus the possibility of an objective evaluation and more intensive data analysis;
a clear distinction between living and dead cells, based on Chl a-fluorescence, this allows a targeted detection of relevant living cells compared to microscopic methods;
a reduced false identification due to the additional taxonomic assignment based on the fluorescence properties;
a statement about the cell size distribution for estimating the growth potential of a species is possible
a statement about localization and amount of chlorophyll per cell for the assessment of the vital state of the cells is possible;
compared to the conventional cytometric evaluation, there is the possibility of taking into account additional morphological information of the measured cells.

### BRIEF DESCRIPTION OF THE DRAWINGS

The invention is explained below in example embodiments based on the associated drawing. It is shown:

FIG. 1 a schematic diagram of an embodiment of an automatic species analysis device; and

FIG. 2 a flow chart for a multi-stage threshold comparison in a method for automatic species analysis according to the invention.

### DETAILED DESCRIPTION OF THE DRAWINGS

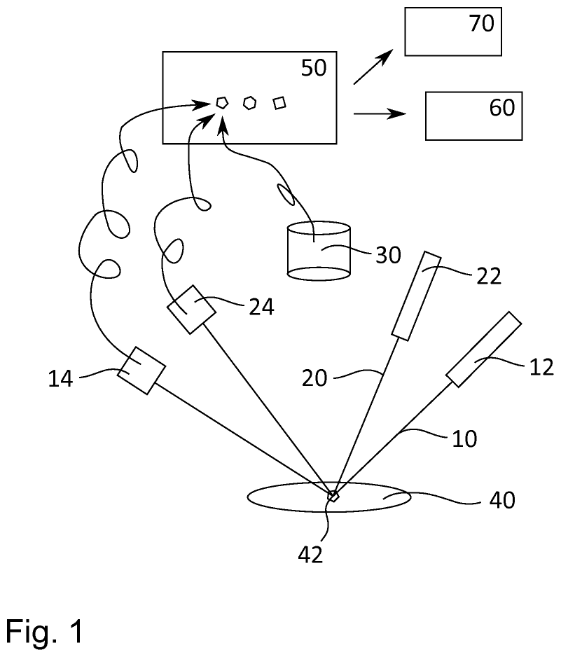

FIG. 1 shows a schematic representation of a embodiment of a device for automatic species analysis. The device comprises a first beam path 10, directed from a first laser radiation source 12 onto a sample area 40, wherein the fluorescence radiation emitted from a sample 42 within the sample area 40 by excitation by the first laser radiation source 12 is collected and fed to a first spectrometer 14 for spectral evaluation; a second beam path 20 is directed from a second laser radiation source 22 to the sample region 40, the fluorescent radiation emitted by the sample 42 within the sample region 40 by excitation by the second laser radiation source 22 being collected and fed to a second spectrometer 24 for spectral evaluation; and a camera 30, configured to take a photographic image of the sample 42 within the sample region 40; the device further comprising a means for automatic species analysis (50), configured to carry out an automatic species analysis from the sample-related results of the spectroscopic evaluation of the first spectrometer (14) and the second spectrometer (24) and the photographic image of the camera (30) by means of a multi-stage threshold value comparison for specific determination parameters stored in an associated database.

Furthermore, the illustration shows a means of archiving 60 connected to the means for automatic species analysis 50, configured to archive the results of the spectroscopic evaluation of the first spectrometer 14 and the second spectrometer 24 as well as the photographic image of the camera 30. The illustration also shows a means for statistical evaluation 70, connected to the means for automatic species analysis 50, configured to perform statistical evaluation of a large number of automatic species analyses on different samples 42.

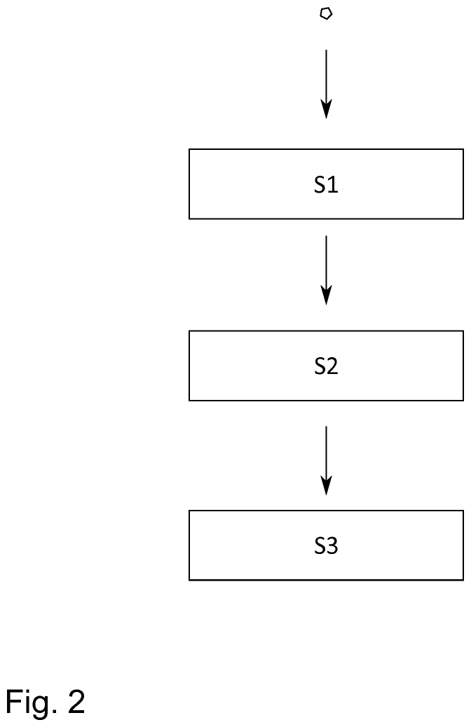

FIG. 2 shows a flow chart for a multi-stage threshold comparison in a method for automatic species analysis according to the invention. A corresponding taxonomic differentiation is carried out in several stages. In the first step S1, a division into spectral groups is carried out on the basis of the sample-related results of the spectroscopic evaluation of the first spectrometer and the second spectrometer (preferably via intensity ratios between the intensity values in different spectral ranges). Subsequently, in a second step S2, a classification at family level is made on the basis of an evaluation of the sample-related photographic image of the camera 30. Corresponding threshold values can be extracted from the photographic images of the camera 30 by means of automatic image recognition methods. Finally, in a third step S3, a classification at species level is made on the basis of a further evaluation of the sample-related photographic image of the camera 30 with regard to further determination parameters and/or taking into account additional determination keys (e.g. corresponding probability statements on the species probability based on the sample origin).

### LIST OF REFERENCE SIGNS

10 First beam path
12 First laser radiation source
14 First spectrometer
20 Second beam path
22 Second laser radiation source
24 Second spectrometer
30 Camera
40 Sample area
42 Sample
50 Means for automatic species analysis
60 Means for archiving
70 Means for statistical evaluation
S1 Step 1
S2 Step 2
S3 Step 3

1. A device for automatic species analysis within a large biological group, comprising:
a sample area;
a first laser radiation source;
a second laser radiation source;
a first spectrometer;
a second spectrometer;
a first beam path, directed from the first laser radiation source onto the sample area, wherein the fluorescence radiation emitted from a sample within the sample area by excitation by the first laser radiation source is collected and fed to the first spectrometer for spectral evaluation;
a second beam path, directed from the second laser radiation source onto the sample area, wherein the fluorescence radiation emitted by the sample within the sample area by excitation by the second laser radiation source is collected and fed to the second spectrometer for spectral evaluation; and
a camera configured to take a photographic image of the sample within the sample area;
wherein the device further comprises a means for automatic species analysis, configured to carry out an automatic species analysis from the sample-related results of the spectral evaluation of the first spectrometer and the second spectrometer and the photographic image of the camera by a multi-stage threshold value comparison for specific determination parameters stored in an associated database.

2. The device according to claim 1, wherein the emission wavelength of the first laser radiation source is between 480 nm and 500 nm.

3. The device according to claim 1, wherein the emission wavelength of the second laser radiation source is between 550 nm and 570 nm.

4. The device according to claim 1, wherein the database of the means for automatic species analysis contains additional determination Keys tor species analysis.

5. The device according to claim 1, the device further comprising a means for archiving, configured to archive the results of the spectral evaluation of the first spectrometer and the second spectrometer and the photographic image of the camera.

6. The device according to claim 1, the device further comprising statistical evaluation means configured to perform statistical evaluation of a plurality of automatic species analyses on different samples.

7. A method for automatic species analysis, comprising the following steps:
Providing of a device according to claim 1;
Introducing a sample into the sample area;
Acquiring a sample-related spectral evaluation of the fluorescence radiation generated with the first spectrometer and the second spectrometer as well as a photographic image of the sample with the camera; and
Evaluating the sample-related results of the spectral evaluation of the first spectrometer and the second spectrometer and of the photographic image of the camera by automatic species analysis, wherein an automatic species analysis is carried out by a multi-stage threshold value comparison for specific determination parameters stored in an associated database.

8. The method according to claim 7, wherein, for the evaluation of the sample-related results of the spectral evaluation of the first spectrometer and the second spectrometer and the photographic image of the camera by automatic species analysis via the threshold value comparison, first of all a division into spectral groups on the basis of the spectral intensity ratios for individual wavelength ranges in the evaluation of the first spectrometer and the second spectrometer, either individually or in combination, and then differentiation at family level is carried out on the basis of an automatic image analysis of the photographic image taken by the camera.

9. The method according to claim 8, wherein further differentiation at species level is subsequently carried out on the basis of an automatic image analysis of the photographic image of the camera and/or by taking into account additional determination keys for species determination.

10. The method according to claim 7, wherein a biovolume determination is additionally carried out by automatic species analysis on the basis of the sample-related results of the spectral evaluation of the first spectrometer and/or the second spectrometer and/or the photographic image of the camera.

11. The method according to claim 7, wherein the sample-related results of the spectral evaluation of the first spectrometer and the second spectrometer and the photographic image of the camera are archived.

12. The method according to claim 7, wherein a statistical evaluation of a plurality of automatic species analyses is carried out on different samples by statistical evaluation.

13. A method for automatic pollen determination according to a method according to claim 7, wherein the ratio between red and blue spectral components, the pollen size, the maximum axial extent of the pollen and/or the geometric asymmetry ratio of the pollen are evaluated as determination parameters.

14. A method for automatic determination of phytoplankton according to a method according to claim 7, wherein excitation of the sample is carried out at an emission wavelength of the first laser radiation source between 480 nm and 500 nm, wherein excitation of the sample is carried out at an emission wavelength of the second laser radiation source between 550 nm and 570 nm for the excitation of phycoerythrin.

15. The method according to claim 14, wherein the cell length, the cell width, the maximum axial extension, the geometric asymmetry ratio of the phytoplankton cells, the cell size, the colony size and/or the size of refractive intracellular structures of the phytoplankton cells are evaluated as determination parameters.

16. A device for automatic species analysis within a large biological group, comprising:
a sample area;
a first laser radiation source;
a second laser radiation source;
a first spectrometer;
a second spectrometer;
a first beam path, directed from the first laser radiation source onto the sample area, wherein the fluorescence radiation emitted from a sample within the sample area by excitation by the first laser radiation source is collected and fed to the first spectrometer for spectral evaluation;
a second beam path, directed from the second laser radiation source onto the sample area, wherein the fluorescence radiation emitted by the sample within the sample area by excitation by the second laser radiation source is collected and fed to the second spectrometer for spectral evaluation; and
a camera configured to take a photographic image of the sample within the sample area;
wherein the device further comprises an analyzer, configured to carry out an automatic species analysis from the sample-related results of the spectral evaluation of the first spectrometer and the second spectrometer and the photographic image of the camera—by a multi-stage threshold value comparison for specific determination parameters stored in an associated database.

17. A method for automatic species analysis, comprising the following steps:
Providing of a device according to claim 16;
Introducing a sample into the sample area;
Acquiring a sample-related spectral evaluation of the fluorescence radiation generated with the first spectrometer and the second spectrometer—as well as a photographic image of the sample with the camera; and
Evaluating the sample-related results of the spectral evaluation of the first spectrometer and the second spectrometer and of the photographic image of the camera by the analyzer, configured to carry out the automatic species analysis, wherein the automatic species analysis is carried out by a multi-stage threshold value comparison for specific determination parameters stored in an associated database.

18. A method for automatic pollen determination according to a method according claim 17, wherein the ratio between red and blue spectral components, the pollen size, the maximum axial extent of the pollen and/or the geometric asymmetry ratio of the pollen are evaluated as determination parameters.

19. A method for automatic determination of phytoplankton according to a method according claim 17, wherein excitation of the sample is carried out at an emission wavelength of the first laser radiation source between 480 nm and 500 nm, wherein excitation of the sample is carried out at an emission wavelength of the second laser radiation source between 550 nm and 570 nm for the excitation of phycoerythrin.

20. A device for automatic species analysis within a large biological group, comprising:
a sample area;
a first laser radiation source;
a second laser radiation source;
a first spectrometer;
a second spectrometer;
a first beam path, directed from the first laser radiation source onto the sample area, wherein the fluorescence radiation emitted from a sample within the sample area by excitation by the first laser radiation source is collected and fed to the first spectrometer for spectral evaluation;
a second beam path, directed from the second laser radiation source onto the sample area, wherein the fluorescence radiation emitted by the sample within the sample area by excitation by the second laser radiation source is collected and fed to the second spectrometer for spectral evaluation; and
a camera configured to take a photographic image of the sample within the sample area;
wherein the device further comprises a circuit, configured to carry out an automatic species analysis from the sample-related results of the spectral evaluation of the first spectrometer and the second spectrometer and the photographic image of the camera—by a multi-stage threshold value comparison for specific determination parameters stored in an associated database.
